(19) United States
(12) Patent Application Publication
(10) Pub. No.: US 2015/0351767 A1
(43) Pub. Date: Dec. 10, 2015

(54) METHODS, COMPOSITIONS, DEVICES AND KITS FOR ANASTOMOSES

(71) Applicant: Boston Scientific Scimed, Inc., Maple Grove, MN (US)

(72) Inventors: Jonathan ZOLL, Brookline, MA (US); Peter J. Pereira, Mendon, MA (US); Michael S.H. Chu, Brookline, MA (US)

(73) Assignee: Boston Scientific Scimed, Inc., Maple Grove, MN (US)

(21) Appl. No.: 14/614,644

(22) Filed: Feb. 5, 2015

Related U.S. Application Data
(60) Provisional application No. 61/936,621, filed on Feb. 6, 2014.

(51) Int. Cl. A61B 17/11; A61B 17/00; A61B 18/24

(52) U.S. Cl. CPC A61B 17/11; A61B 18/24; A61B 17/00491; A61B 2017/1132; A61B 2017/005

(57) ABSTRACT

The present disclosure is directed to methods, compositions, devices and kits which pertain to the attachment of one body conduit portion to another body conduit portion by application of an energy source to body conduit portions in the presence of a bonding material.

### STATEMENT OF RELATED APPLICATION

This application claims the benefit of U.S. Provisional Application Ser. No. 61/936,621, filed Feb. 6, 2014 and entitled “METHODS, COMPOSITIONS, DEVICES AND KITS FOR ANASTOMOSES,” the entire disclosure of which is hereby incorporated by reference in its entirety.

### FIELD OF THE INVENTION

The present invention pertains to methods, compositions, devices and kits that are useful in forming anastomoses.

### BACKGROUND

Many body conduits are generally cylindrical in configuration and have a generally circular cross-section. The surgical attachment of one body conduit to another is commonly referred to as an anastomosis.

As one specific example, urethral anastomosis is a procedure that often occurs after the prostate is removed and the urethra is split into two pieces that need to be reconnected. For example, in radical prostatectomy, the surgeon removes all or most of the patient's prostate. Because the urethra travels through the prostate immediately before reaching the bladder, a portion of the urethra is removed in the surgery. In order to restore proper urinary functions, the bladder neck and the urethral stump are reconnected.

Existing procedures often involve connecting the two ends together using sutures. This is a difficult process as the urethra is not rigid and it is hard to keep it in place as the surgeon threads the suture. Additionally sutures do not guarantee a watertight seal which can be problematic for the urethra.

### SUMMARY OF THE INVENTION

The present invention relates to methods, compositions, devices and kits for anastomoses.

In accordance with some aspects of the present disclosure, anastomosis bonding components are provided which comprise a bonding material. The anastomosis bonding components are adapted to bond and/or seal a first body conduit portion to a second body conduit portion upon exposure to energy from an energy source.

Other aspects of the present disclosure are directed to methods of performing anastomosis procedures. The methods comprise: (a) placing a bonding component comprising a bonding material in contact with a first body conduit portion and a second body conduit portion and (b) applying energy from an energy source onto the bonding component, the first body conduit portion and the second body conduit portion, such that the bonding material is activated and the first and second body conduit portions become directly or indirectly bonded to one another, and such that a fluid seal is established between the first and second body portions.

Still other aspects of the present disclosure are directed to devices and kits performing anastomosis procedures.

These and other aspects, as well as various embodiments and advantages of the present invention will become immediately apparent to those of ordinary skill in the art upon review of the Detailed Description and claims to follow.

### BRIEF DESCRIPTION OF THE DRAWINGS

FIGS. 1A-1F are schematic illustrations of a procedure for forming a urethral anastomosis, in accordance with an embodiment of the present invention, wherein FIG. 1A illustrates the procedure at a first point in time, FIG. 1B illustrates the procedure at a second point in time, FIG. 1C illustrates the procedure at a third point in time, FIG. 1D illustrates the procedure at a fourth point in time, FIG. 1E illustrates the procedure at a fifth point in time, and FIG. 1F illustrates the procedure at a sixth point in time.

FIGS. 2A-2F are schematic illustrations of a procedure for forming a urethral anastomosis, in accordance with an embodiment of the present invention, wherein FIG. 2A illustrates the procedure at a first point in time, FIG. 2B illustrates the procedure at a second point in time, FIG. 2C illustrates the procedure at a third point in time, FIG. 2D illustrates the procedure at a fourth point in time, FIG. 2E illustrates the procedure at a fifth point in time, and FIG. 2F illustrates the procedure at a sixth point in time.

FIG. 3A is a schematic illustration of a hollow filamentous tube of bonding material in a first configuration and FIG. 3B is a schematic illustration of a hollow filamentous tube of bonding material in a second configuration, in accordance with an embodiment of the present invention.

FIG. 4 is a schematic illustration of a slotted hollow tube of bonding material, in accordance with another embodiment of the present invention.

FIG. 5A is a schematical illustration of a bonding-material-containing support structure, in accordance with another embodiment of the present invention. FIG. 5B is a cross-sectional view of FIG. 5A, taken along line B-B. FIG. 5C is a schematic illustration of a support structure, in accordance with another embodiment of the present invention.

FIGS. 6A-6K are schematic illustrations of a procedure for forming a urethral anastomosis, in accordance with another embodiment of the present invention, wherein FIG. 6A illustrates the procedure at a first point in time, FIG. 6B illustrates the procedure at a second point in time, FIG. 6C illustrates the procedure at a third point in time, FIG. 6D illustrates the procedure at a fourth point in time, FIG. 6E illustrates the procedure at a fifth point in time, FIG. 6F illustrates the procedure at a sixth point in time, FIG. 6G illustrates the procedure at a seventh point in time, FIG. 6H illustrates the procedure at an eighth point in time, FIG. 6I illustrates the procedure at a ninth point in time, FIG. 6J illustrates the procedure at a tenth point in time, and FIG. 6K illustrates the procedure at an eleventh point in time.

FIGS. 7A-7F are schematic illustrations of a procedure for forming a urethral anastomosis, in accordance with yet another embodiment of the present invention, wherein FIG. 7A illustrates the procedure at a first point in time, FIG. 7B illustrates the procedure at a second point in time, FIG. 7C illustrates the procedure at a third point in time, FIG. 7D illustrates the procedure at a fourth point in time, FIG. 7E illustrates the procedure at a fifth point in time, and FIG. 7F illustrates the procedure at a sixth point in time.

FIG. 8A is a schematic illustration of a component useful for forming a urethral anastomosis, in accordance with an embodiment of the present invention and FIG. 8B is a schematic illustration of a component useful for forming a urethral anastomosis, in accordance with another embodiment of the present invention.

FIGS. 9 is a schematic illustration of a balloon catheter useful for forming a urethral anastomosis, in accordance with an embodiment of the present invention. FIG. 10 is a schematic illustration of balloon catheter useful for forming a urethral anastomosis, in accordance with another embodiment of the present invention. FIG. 11 is a schematic illustration of balloon catheter useful for forming a urethral anastomosis, in accordance with yet another embodiment of the present invention.

FIG. 12 is a schematic cross-sectional illustration of a double balloon catheter useful for forming a urethral anastomosis, in accordance with an embodiment of the present invention.

FIG. 13 is a schematic partial cross-sectional illustration of a procedure for forming a urethral anastomosis, in accordance with yet another embodiment of the present invention.

FIGS. 14A-14G are schematic illustrations of a procedure for forming an esophageal anastomosis, in accordance with still another embodiment of the present invention, wherein FIG. 14A illustrates the procedure at a first point in time, FIG. 14B illustrates the procedure at a second point in time, FIG. 14C illustrates the procedure at a third point in time, FIG. 14D illustrates the procedure at a fourth point in time, FIG. 14E illustrates the procedure at a fifth point in time, FIG. 14F illustrates the procedure at a sixth point in time, and FIG. 14G illustrates the procedure at a seventh point in time.

### DETAILED DESCRIPTION

A more complete understanding of the present invention is available by reference to the following detailed description of numerous aspects and embodiments of the invention. The detailed description which follows is intended to illustrate but not limit the invention.

In various beneficial embodiments, tissue bonding technology is used as a technique for attaching a first body conduit portion to a second body conduit portion, for example, a first portion of a urethra (e.g., the urethral stump) to a second portion of the urethra (e.g., the bladder neck). This is achieved by joining the first and second body conduit portions and placing a bonding material in intimate association with the area where the first and second body conduit portions contact one another. Subsequently, energy is applied from an energy source to activate the bonding material and bond and/or seal the first body conduit portion to the second body conduit portion. In this way, the present disclosure provides for the attachment of one body conduit portion to another body conduit portion without the use of sutures, staples or other mechanical fasteners in some embodiments, or as a supplement to such fasteners in other embodiments.

Different energy sources may be used for tissue bonding, depending on the mechanism for tissue bonding that is employed. The energy source may be, for example, a source of heat or light, such as a laser or a light-emitting diode (LED). Infrared and near-infrared laser sources include carbon dioxide (CO₂), thulium—holmium—chromium, holmium, thulium, and neodymium rare-earth-doped-garnets (THC:YAG, Ho:YAG, Tm:YAG, and Nd:YAG, respectively), and gallium aluminum arsenide diode (GaAlAs) lasers, among others. Visible sources include potassium-titanyl phosphate (KTP) frequency-doubled Nd:YAG, and argon lasers, among others. Other energy sources include radiofrequency sources (e.g., a microwave source), radiation sources (e.g., x-ray radiation, gamma radiation, etc.), or a locally produced plasma. Argon plasmas are currently employed in various medical applications, including argon beam coagulators, which ionize argon gas to form an argon plasma and then use the plasma to deliver thermal energy to nearby tissue. In the present disclosure, an argon beam may be used as a source of heat for tissue bonding. Other energy sources include radiation (e.g., x-ray radiation, gamma radiation, etc.).

In certain embodiments, the energy source is a handheld energy source.

In certain embodiments, the energy source is provided in a stand-alone unit. In other embodiments, the energy source is combined with another device. For example, the energy source may be combined with a surgical device, thereby creating a single unit that can position and bond and/or seal the tissue.

In some embodiments, the energy source is connected to a control unit, which controls the energy emitting from the energy source. Preferably, the amount of energy is sufficient to activate the bonding material without significantly damaging the tissue. In some embodiments, the control unit is designed to accept user input (e.g., via physical buttons, touchscreen, etc.), thereby allowing treatment parameters to be set by a health care provider.

In some embodiments, the energy source is controlled without the use of a sensor (e.g., based on the experience of the surgeon or based on a suitable energy output algorithm). In other embodiments, a sensor is used in conjunction with the energy source to provide feedback regarding the amount of energy being directed to the bonding site, and this feedback can be used to adjust the energy source output. For example, in certain embodiments, the sensor is a temperature sensor which detects the amount of heat at the bonding site. In these embodiments, suitable software can be employed to adjust the output of the energy source based on input from the temperature sensor. The sensor may be provided, for example, in the same device as the energy source or in a device that is different from the device containing the energy source. The sensor may be provided, for example, in a surgical device (either with or without the energy source) that is used to manipulate the body conduit portions into a suitable position for anastomosis.

A variety of bonding materials can be used in conjunction with the present disclosure.

In this regard, laser tissue soldering processes are known in the surgical art whereby tissue is bonded by applying a solder (commonly, a biological polymer) to the tissue after which a laser is used to activate the solder and form a bond. Without wishing to be bound by theory, it has been reported that the mechanism of laser tissue soldering appears to include a heating-induced protein denaturation-renaturation process. See, e.g., B. Forer et al., Laryngoscope 116: June 2006, 1002-1006.

In some embodiments, solder materials are used in the present disclosure as bonding materials to bond and/or seal a first body conduit portion (e.g., a urethral stump) to a second body conduit portion (e.g., a bladder neck). For example, heat energy may be applied to a solder material while it is in contact with the body conduit portions in an area where the body conduit portions are placed in contact with one another such that the body conduit portions become bonded to one another. The bonded conduits creates a sealed conduit that ensures that fluid (e.g., urine, blood, etc.) does not leak through the bonded junction. As indicated above, beneficial energy sources for the application of heat include light sources (e.g., lasers, etc.), radiofrequency sources (e.g., microwave sources, etc.) and plasma sources (e.g., argon beams, etc.), among others.

Particularly beneficial solder materials have a relatively low activation temperature and are bio-absorbable. Over time the solder may be bioabsorbed, leaving only adjoined tissue behind.

Specific solder materials for use in conjunction with the present disclosure include solders of biological origin and synthetic solders. Examples of solders of biological origin include those based on biological polymers, for example, polypeptides including nano-peptides and proteins such as albumin, collagen, elastin and fibrin, protein derivatives, as well as polysaccharides including chitosan, among others. Examples of solders of synthetic origin include polylactide, polyglycolide, poly(glycerol sebacate acrylate), and poly(lactide-co-glycolide). In some embodiments, two, three, four or more solder materials such as those described above are employed. Specific examples include a combination of albumin and collagen, a combination of albumin and chitosan, a combination of collagen and chitosan, and a combination of albumin, collagen, and chitosan, among many other possible combinations.

In some embodiments, at least one energy absorber is used within the solder material to enhance heating efficiency and/or heat distribution within the solder material. Energy absorbers include chromophores, for example, light-specific dyes such as indocyanine green (ICG), fluorescein, basic fuchsin, and fen, nano-gold (e.g., gold nanorods, gold nanoshells, gold nanocages, etc.), SPIONs (superparamagnetic iron oxide nanoparticles), and silica nanoparticles, among other materials. Specific examples include ICG-doped albumin, fluorescein-dye-doped albumin, and nano-gold-doped albumin, among many others.

Photochemical tissue bonding processes are known the surgical art. These processes take advantage of the photochemical reactions that occur at intimately associated tissue surfaces, which are stained with a photosensitizing dye (e.g., dyed tissue surfaces which are placed in contact with one another). Without wishing to be bound by theory, it is believed that the dye absorbs photons of visible radiation and promotes the formation of covalent bonds between molecules on the adjacent tissue surfaces. For example, reactive species that are produced upon light activation of the dye can react with potential electron donors and acceptors such as amino acids in proteins (e.g., tryptophan, tyrosine, cysteine, and so forth). In this regard, photochemical methods have been reported to form crosslinks in collagen type I molecules. See, Barbara P. Chan et al., Journal of Surgical Research 108, 77-84 (2002).

In certain aspects of the present disclosure, photosensitizing dyes are used to bond and/or seal a first body conduit portion to a second body conduit portion, for example, by the application of light of a suitable wavelength to a photosensitizing dye in intimate association with the body conduit portions (e.g., a photosensitizing dye positioned at an interface where the body conduit portions are brought into contact with one another), such that the first and second body conduit portions are bonded and/or sealed to one another. A light-emitting energy source such as a low-power laser or light-emitting diode (LED) may be used for this purpose, among others.

In some embodiments, a photosensitizing dye is combined with a solder material (e.g., a biological solder material, including those set forth above, among others). For example, a photosensitizing dye may be admixed with a solder material or coated on a surface of a solder material.

Specific examples of photosensitizing dyes include one or more of the following, among others: xanthene dyes such as rose bengal, methylene blue and fluorescein, riboflavin dye (e.g., riboflavin-5-phosphate), lumichrome dye, lumiflavin dye, Reactive Black 5, thiazine dye, erythrosine, N-hydroxypyridine-2-(1H)-thione (N-HTP), protoporphyrin I through protoporphyrin IX, coproporphyrins, uroporphyrins, mesoporphyrins, hematoporphyrins and sapphyrins, chlorophylis, e.g., bacteriochlorophyll A, Photofrin®, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series (e.g., protoporphyrin I through protoporphyrin IX, coproporphyrins, uroporphyrins, mesoporphyrins, hematoporphyrins and sapphyrins), chlorins, chlorin e6, mono-1-aspartyl derivative of chlorin e6, di-1-aspartyl derivative of chlorin e6, tin(IV) chlorin e6, meta-tetrahydroxphenylchlorin, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, chlorophylis, bacteriochlorophyll A, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid, benzonaphthoporphyrazines, cationic imminium salts, tetracyclines, lutetium texaphyrin, texaphyrin, tin-etio-purpurin, porphycenes, benzophenothiazinium, eosin, erythrosin, cyanines, merocyanine 540, selenium substititued cyanines, flavins, riboflavin, proflavin, quinones, anthraquinones, benzoquinones, naphthaldiimides, naphthalimides, victoria blue, toluidine blue, dianthroquinones (e.g., hypericin), fullerenes, rhodamines and photosensitive derivatives thereof.

An advantage of using light rather than heat is that there is less risk of causing damage to the tissue (cell death) from heat. Another advantage of using light, rather than heat, to achieve tissue bonding is that complications due to uneven heat distribution can be reduced or eliminated.

In addition, the use of wavelength-specific absorbers such as chromophores enables differential absorption between the chromophore-containing regions and surrounding tissue. One advantage is a selective absorption of radiation by the target, without the need for a precise focusing. Moreover, lower power levels may be used because of the increased absorption of chromophore-containing regions, leading to reduced tissue damage.

Various aspects of the present disclosure will now be described in conjunction with the drawings. It should be noted that, although urethral and esophageal anastomoses are exemplified in the following paragraphs, other types of anastomoses may be formed in an analogous fashion.

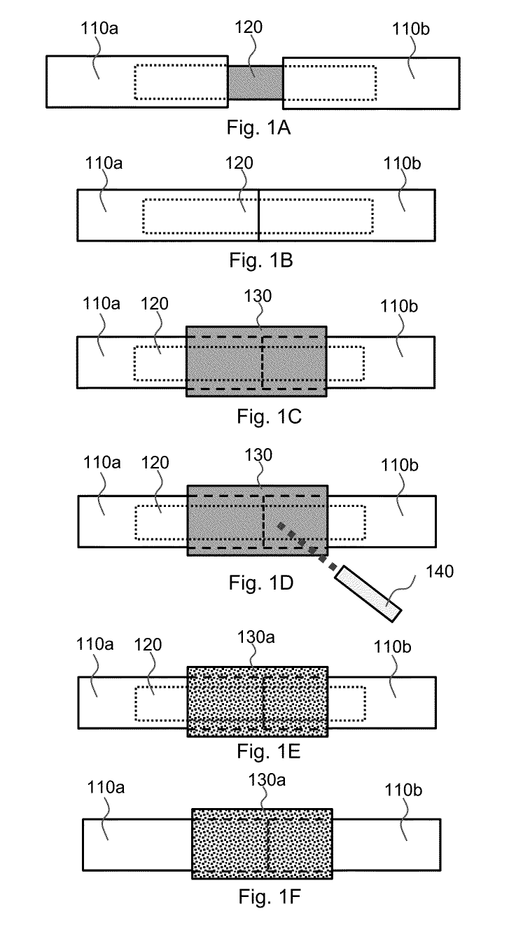

Turning now to FIGS. 1A-1F, a method for forming an anastomosis in accordance with an embodiment of the invention is schematically illustrated. During various surgical procedures (e.g., a radical prostatectomy, etc.), the urethra of a patient may be severed into a first urethral portion 110a (e.g., a urethral stump) and a second urethral portion 110b (e.g., a bladder neck), which are to be joined together to re-establish a path for urine flow. As shown in FIG. 1A the first and second urethral portions 110a, 100b may be positioned over a support structure 120 which supports the first and second urethral portions 110a, 110b during the anastomosis procedure, for example, keeping them from collapsing. The first and second urethral portions 110a, 110b are fitted over the support structure 120 and brought into contact with one another as shown in FIG. 1B. At some point prior to attaching the first and second urethral portions 110a, 110b, bonding material is intimately associated with the first and second urethral portions 110a, 110b where they come into contact with each other. For example, a bonding material may be supplied in a fluid form (e.g., a liquid, gel or paste form) at an interface between the first and second urethral portions 110a, 110b. Alternatively or in addition, a bonding material may be supplied in in the form of a ring or hollow tube of bonding material 130 which surrounds and covers the interface where the first and second urethral portions 110a, 110b meet.

A ring or hollow tube of bonding material 130 may be established, for example, by applying a gel or paste of bonding material around the circumference of the first and second urethral portions 110a, 110b or by wrapping a ribbon/tape/film of bonding material one or more times around the circumference of the first and second urethral portions 110a, 110b.

A ring or hollow tube of bonding material 130 may also be established by positioning a bonding material member in the form of a tube (e.g., a sleeve) around the first and second urethral portions 110a, 110b. For example, as shown in FIG. 4, a sheet of bonding material 130 may be provided with a shape memory in the form of a tube (e.g., where the edges 130e of the sheet nearly meet, meet or overlap one another, thereby forming the tube). When placing the bonding material 130, an opening force is applied to move the edges 130e apart and create a gap 130g as shown in FIG. 4. The gap 130g is spread sufficiently wide to allow the bonding material 130 to be positioned around the first and second urethral portions, after which the opening force is removed, and the bonding material 130 forms a tubular shape that contacts the surface of the first and second urethral portions 110a, 110b.

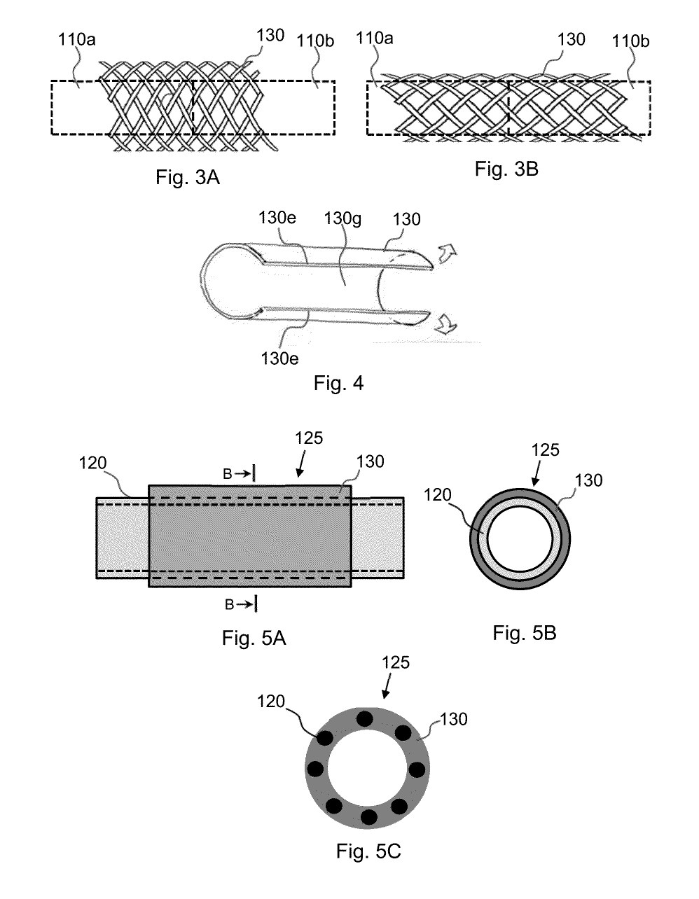

In other embodiments, bonding material 130 may be in a form of an expanded diameter preformed tube of bonding material 130, whose diameter can be reduced to a contracted diameter after being placed around the first and second urethral portions 110a, 110b. In this regard, one of the urethral portions 110a, 110b may be inserted through the expanded diameter preformed tube of bonding material 130 prior to bringing the urethral portions 110a, 110b into contact with one another. In one example shown in FIGS. 3A and 3B, the expanded diameter preformed tube of bonding material 130 may be formed from braided filaments of bonding material analogous to a Wallstent™ or “Chinese finger trap”. The braided filaments may be of various cross-sections including circular, oval, polygonal, and so forth. The braided filaments may be flat, for instance in the form of ribbons/strips. Once placed around the first and second urethral portions 110a, 110b as shown in FIG. 3A, the preformed tube of bonding material 130 may be reduced in diameter to contact to the surface of first and second urethral portions 110a, 110b as shown in FIG. 3B. This reduction in diameter will be accompanied by an increase in length as shown in FIGS. 3A-3B. In other embodiments, the preformed tube of bonding material 130 may be formed form a heat-shrinkable material that reduces the diameter upon application of heat (e.g., either independent of or in conjunction with the application of energy for tissue bonding).

Returning to FIGS. 1A-1F, after a ring or hollow tube of bonding material 130 has been positioned around the first and second urethral portions 110a, 110b as shown in FIG. 1C, an energy source 140 is used to supply energy (e.g., heat and/or light) to the bonding material 130 disposed around the first and second urethral portions 110a, 110b, with the result being that the bonding material is activated (forming activated bonding material 130a) and the first and second urethral portions 110a, 110b are bonded and sealed to one another as shown in FIG. 1E. The support structure 120 is then removed creating a leak-free conduit as shown in FIG. 1F.

In the preceding embodiment, the support structure may be formed of any suitable material, for example a polymeric, ceramic or metallic material, among others, which has sufficient rigidity to support the first and second urethral portions during the procedure. For example, the support structure may be in the form of an inflated cylinder which is deflated and withdrawn from the urethra after the first and second urethral portions have been attached. As another example, the support structure may be in the form of a hollow tubular segment (stent) that is withdrawn from the urethra after the first and second urethral portions have been attached. As yet another example, the support structure may be in the form of a catheter tube that is temporarily inserted through the first and second urethral portions and removed from the urethra after the first and second urethral portions have been attached. For instance, a catheter such as a Foley catheter can be inserted through the urethra and up to the bladder before removal of a subject's prostate. The presence of the catheter assists in connecting the two ends of the urethra together. An open sleeve (e.g., like that of FIG. 4, among others) may then be placed after the two ends are drawn together, among other possibilities.

In certain embodiments, the support structure may be in the form of a hollow tubular segment (stent) that degrades over time, thereby providing support during at least the initial stages of the healing process. Degradable materials include degradable polymeric, ceramic or metallic materials, among others.

In still other embodiments, the support structure may comprise a bonding material as described herein. In this regard, the support structure may be formed entirely of bonding material. For instance, the support structure may be in the form of a hollow tubular segment (e.g., stent) that is formed entirely from bonding material. The hollow tubular segment may have the ability to expand or contract based on the tubular segment stent structure and covering.

The support structure may also be partially formed from a bonding material. For instance, the support structure may comprise a layer of bonding material disposed over an outer surface of an underlying support structure. The layer of bonding material may cover all or only a portion of the underlying support structure. In these embodiments, the underlying support structure may be formed of any suitable material, for instance, a biostable or biodegradable polymeric, ceramic or metallic material, among others.

For example, the underlying support structure may be in the form of a catheter tube that is partially coated with bonding material, which is temporarily inserted through the first and second urethral portions and removed from the urethra after the first and second urethral portions have been attached. In other examples, the underlying support structure may be in the form of an inflated cylinder that is at least partially coated with bonding material, which is deflated and withdrawn from the urethra after the first and second urethral portions have been attached, or in the form of a hollow tubular segment (stent) that is at least partially coated with bonding material, which is withdrawn from the urethra after the first and second urethral portions have been attached. In still other examples, the support structure may be in the form of a hollow tubular segment (e.g., stent) that is at least partially coated with bonding material, which degrades over time, after the first and second urethral portions have been attached, thereby providing support during the initial stages of the healing process.

One embodiment of such a structure is schematically illustrated in FIGS. 5A and 5B, which show a bonding-material-containing support structure 125 in accordance with the present invention. FIG. 5B is a cross-sectional view of FIG. 5A, taken along line B-B. The support structure 125 comprises an underlying support structure 120 (e.g., stent, catheter, inflatable cylinder, etc.) having an outer layer of bonding material 130. The underlying support structure 120 may be partially or completely coated with the bonding material. In the embodiment shown, the underlying support structure 120 is only partially coated with the bonding material 130 (i.e., on the outer surface of the underlying support structure 120). In another embodiment shown in FIG. 5C, the support structure 125 comprises an underlying support structure 120 in the form of a stent, which is completely covered (i.e., embedded) in bonding material 130.

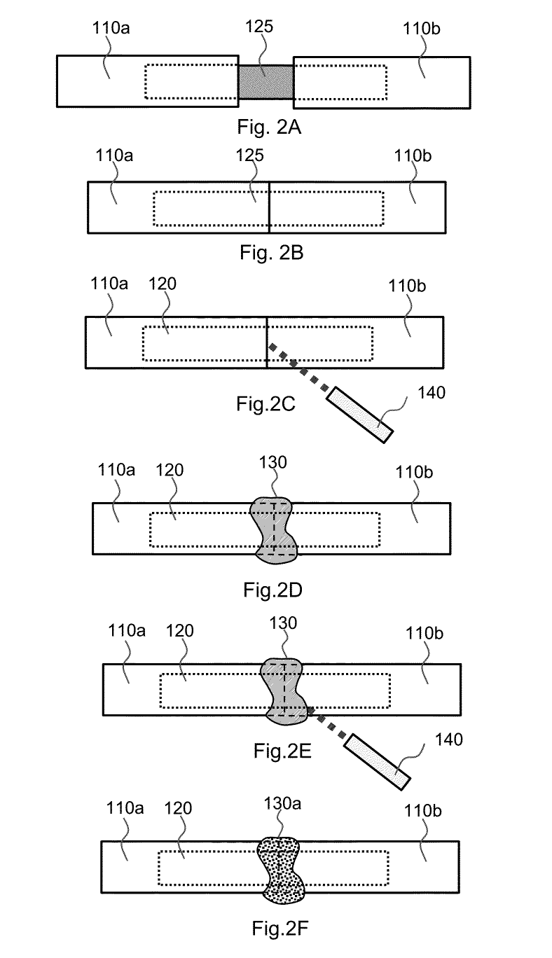

A further procedure which makes use of a bonding-material-containing support structure 125 will now be described in conjunction with FIGS. 2A-2F. As shown in FIG. 2A, first and second urethral portions 110a, 100b may be positioned over a bonding-material-containing support structure 125 which supports the first and second urethral portions 110a, 110b during the anastomosis procedure. The first and second urethral portions 110a, 110b are fitted over the support structure 125 and brought into contact with one another as shown in FIG. 2B.

An energy source 140 is used to supply energy (e.g., heat and/or light) to the first and second urethral portions 110a, 110b and the bonding material in the support structure 125, as shown in FIG. 2C, with the result being that the bonding material is activated and the first and second urethral portions 110a, 110b are bonded and/or sealed to one another. Although the energy source 140 is shown as being external to the ureter in FIG. 2C, energy may be directed to the support structure 125 within the ureter as well (e.g., where the support structure 125 is in the form of a stent that is completely formed from bonding material, or the bonding material is coated on an underlying stent support structure that is transparent to energy from the energy source 140, or the support contains apertures allowing for the transmission of energy from the energy source to the bonding material, for example, as shown in FIG. 5C).

In a further optional step, a ring or hollow tube of bonding material 130 is placed at the interface between the first and second urethral portions 110a, 110b as shown in FIG. 2D. (While is noted that the bonding material 130 in FIG. 2D is applied in the form of a fluid, various solid forms may be applied as described above.) Subsequently, as shown in FIG. 2E, an energy source 140 is used to supply energy (e.g., heat and/or light) to the bonding material 130 disposed on the first and second urethral portions 110a, 110b, with the result being that the bonding material is activated (forming activated bonding material 130a) and the first and second urethral portions 110a, 110b are further bonded and/or sealed to one another, as illustrated in FIG. 2F.

As seen from the foregoing, bonding material may be applied internally and/or externally to the urethra. Similarly, energy may be applied internally and/or externally to the urethra.

In certain embodiments, a support structure (either having a bonding material or not having a bonding material) may be provided with surface features that assist in holding the ureter portion on the support. For example, the support structure may be a stent whose surface comprises teeth (e.g., like the teeth of a rasp) which are configured to allow each portion of the ureter to be fitted onto the support and advanced with relative ease toward the center of the support. However, on attempted retraction of the tissue from the support, the teeth engage the tissue and resist movement. The teeth will, in general, be biased toward the center of the stent. In these embodiments the stent may be, for example, formed from the bonding material or comprised of a coating of bonding material on a degradable underlying structure.

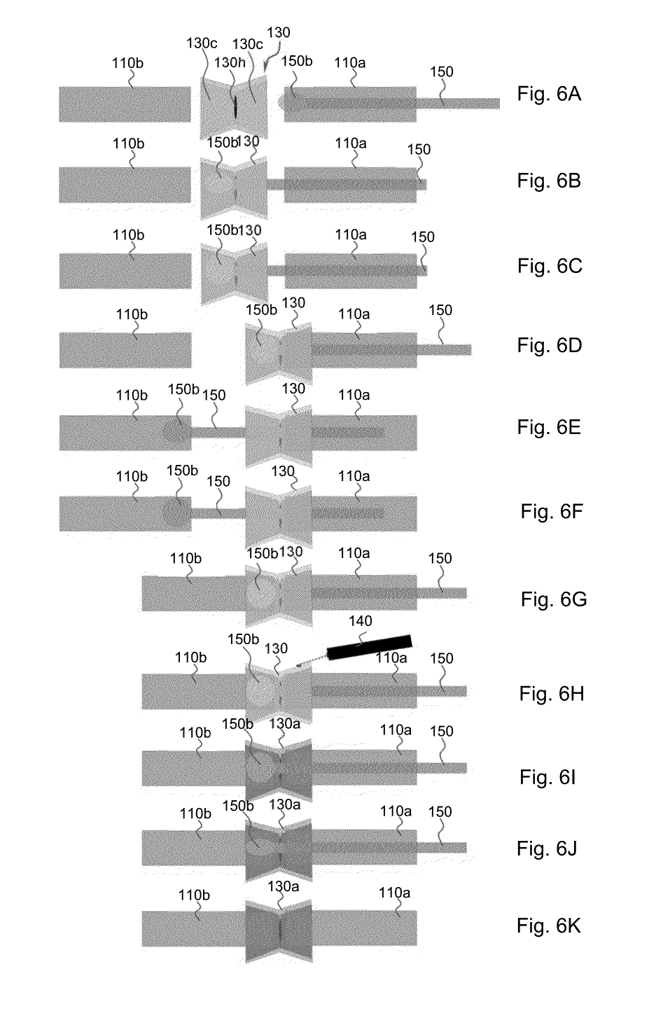

In some embodiments, an expandable device such as a balloon (e.g., a balloon associated with a balloon catheter) may be used in the bonding procedure. As an example, turning now to FIGS. 6A-6K, a balloon 150b at a distal end of a catheter 150 is inserted through the urethral exit and advanced until the balloon 150b emerges from a first urethral portion 110a (e.g., a urethral stump) into the gap between the urethral portions 110a,110b. The balloon 150b is further advanced through a bonding-material-containing sleeve 130 as shown in FIG. 6B. Then the balloon 150b is expanded (inflated) as shown FIG. 6C and pulled back such that the sleeve 130 slides over the first urethral portion 110a as shown in FIG. 6D. The balloon 150b is then contracted (deflated) and directed into the second urethral portion 110b (e.g., a bladder neck) as shown in FIG. 6E. The balloon 150b is expanded (inflated) such that it engages the second urethral portion 110b as shown in FIG. 6F, and then drawn back, pulling the second urethral portion 110b inside the sleeve as shown in FIG. 6G. The sleeve 130 is then irradiated by an energy source 140 as shown in FIG. 6H with the result being that the bonding material in the sleeve 130 is activated, forming activated bonding material 130a as shown in FIG. 61. The balloon 150b is then contracted (deflated) as shown in FIG. 6J and the catheter 150 removed from the urethra, completing the process as shown in FIG. 6K.

In the embodiment shown in FIGS. 6A-6K, the sleeve 130 is a generally tubular structure, in particular, a dual-chambered “bowtie” design having two tapered open chambers 130c with a hole 130h for the catheter to fit through (see FIG. 6A). The balloon 150b only needs to expand to a size that is larger than the sleeve hole 130h and large enough to engage the second urethral portion 110b. The chambers 130c are partial conic sections as shown, however, other geometries are possible (e.g., partial pyramidal sections, hemispheres, etc.). The sleeve 130 may be formed, for example, of a material which can be compressed for delivery through the first urethral portion 110a and which self-expands after emerging from the first urethral portion 110a at the surgical site. The sleeve 130 may also be formed of a material that enables the ends of the sleeve 130 to expand outwardly for easier urethral fit.

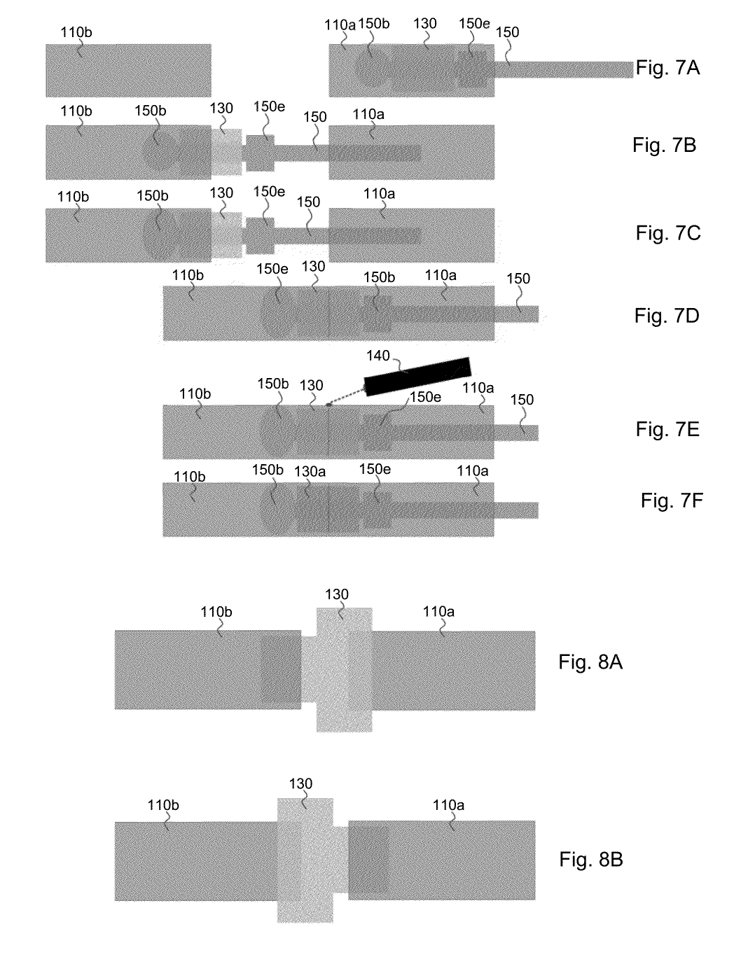

Another embodiment will now be described in conjunction with FIGS. 7A-7F.

Turning now to FIG. 7A, an expandable device such as a balloon 150b of a balloon catheter 150 is inserted through the urethral exit and advanced. A bonding-material-containing sleeve 130 is disposed over the catheter 150 just proximal to the balloon 150b and just distal to an enlarged portion of the catheter 150e (e.g., a step) such that the sleeve 130 is prevented from longitudinal motion by the balloon 150b and the expanded portion 150e. The catheter 150 carrying the sleeve 130 is advanced such that the balloon 150b travels through first urethral portion 110a, across the gap between the urethral portions 110a,110b, and into the second urethral portion 110b, such that the balloon and a distal portion of the sleeve 130 are positioned in the second urethral portion 110b as shown in FIG. 7B. The balloon 150 is expanded (inflated) such that it engages the second urethral portion 110b as shown in FIG. 7C, and the catheter 150 is then drawn back, pulling the proximal portion of the sleeve 130 back into the first urethral portion 110a as shown in FIG. 7D. In certain embodiments (not shown) the balloon may be deflated, retracted to the center of the sleeve and expanded to better engage the sleeve with the urethra. Once the sleeve 130 is positioned inside the urethra at the interface between the first and second urethral portions 110a,110b as shown in FIG. 7D, the sleeve 130 is irradiated by an energy source 140 as shown in FIG. 7E, with the result being that the bonding material in the sleeve 130 is activated, forming activated bonding material 130a as shown in FIG. 7F. Although the energy source 140 is external to the urethra in the embodiment shown in FIG. 7E, an internal energy source 140 may be used internally as previously discussed. The balloon is then contracted (deflated) and the catheter is removed from the urethra. The balloon 150b may have three steps of inflation: (a) completely deflated such that the balloon can pass through the sleeve 130, (b) an intermediate level wherein the balloon is inflated just enough for the sleeve 130 to be unable to slip off the catheter during delivery of the balloon 150b, and (c) a highest level of inflation wherein the balloon 150b is able to engage the urethra from the inside.

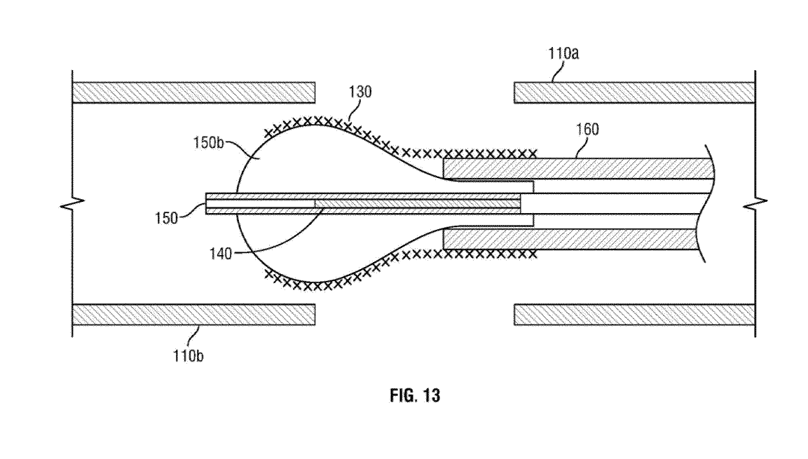

A related embodiment will now be described with reference to the partial cross-sectional illustration shown in FIG. 13. In this method, a bonding material-containing expandable sleeve 130 (e.g., expandable stent such as a braid or mesh stent that is coated with or embedded within a bonding material, such as a solder) can be loaded partially onto a balloon 150b and partially onto a retractable sheath 160 as shown in FIG. 13. The balloon 150b, associated catheter shaft 150, sheath 160 and expandable sleeve 130 are advanced through the first urethral portion 110a, across the gap between the urethral portions 110a,110b, and partially into the second urethral portion 110b, such that a distal portion of the balloon 150b and a distal portion of the expandable sleeve 130 are positioned in the second urethral portion 110b as shown in FIG. 13. An inflation fluid is introduced into the balloon 150 such that the distal portion of the balloon 150, which is not in the sheath 160, expands, thus also expanding the distal portion of the expandable sleeve 130 such that the distal portion of the balloon 150b and distal portion of the expandable sleeve 130 engage the second urethral portion 110b. A retractable energy source 140 within the catheter shaft 150 is then energized to activate the bonding material in the distal portion of the expandable sleeve 130, thereby bonding the distal portion of the expandable sleeve 130 to the urethral portion 110b. In this particular embodiment, this step requires the catheter shaft 150 and surrounding balloon 150b to be at least partially transparent to the energy emitted from the energy source 140. The balloon 150b, catheter shaft 150, sheath 160 and expandable sleeve 130 are then drawn back, pulling the proximal portion of the expandable sleeve 130 and proximal portion of the balloon 150 into the first urethral portion 110a (which also draws the second urethral portion 110b and the first urethral portion 110a toward one another). At this point the sheath 160 is withdrawn, relative to the balloon 150 and the expandable sleeve 130, allowing the proximal portion of balloon 150 (which emerges from the sheath 160) to expand. This also expands the proximal portion of the expandable sleeve 130, such that the proximal portion of balloon 150 and the proximal portion of the expandable sleeve 130 engage the first urethral portion 110a. The energy source 140 within the catheter shaft 150 is then retracted to a position where the energy source 140 activates the bonding material in the proximal portion of the expandable sleeve 130, thereby bonding the proximal portion of the expandable sleeve 130 to the urethral portion 110a. The balloon 150b is then deflated, and the catheter shaft 150, balloon 150b, sheath 150 and energy source 140 are withdrawn from the patient.

The sleeve 130 is positioned externally to the urethra in FIGS. 6A-6K, while the sleeve 130 is positioned internally to the urethra in FIGS. 7A-7F and 13. In another embodiment shown in FIG. 8A, the sleeve 130 is positioned externally to a first urethral portion 110a (e.g., a urethral stump) internally to a second urethral portion 110b (e.g., a bladder neck). In yet another embodiment shown in FIG. 8B, the sleeve 130 is positioned internally to a first urethral portion 110a (e.g., a urethral stump) externally to a second urethral portion 110b (e.g., a bladder neck). The sleeves in FIGS. 8A-8B may be positioned using techniques analogous to those described in conjunction with FIGS. 6A-6K and 7A-7F.

Although a catheter with an expandable balloon is used in the preceding embodiments, in other embodiments a catheter may be employed which has an expandable cage or stent.

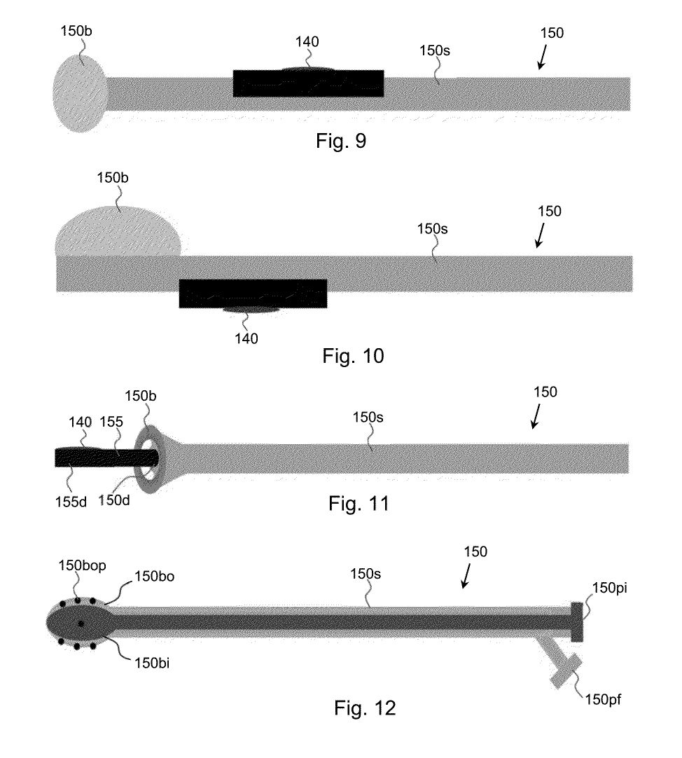

As previously noted, in some embodiments, bonding material is activated through the use of an energy source that is positioned internally relative to the urethra. Turning to FIGS. 9 and 10, in each illustration, a catheter 150 is shown, which includes a catheter shaft 150s, a balloon 150b and an energy source 140 that is integrated into the catheter 150. The balloon 150b in the catheter 150 of FIG. 10 is an asymmetrical balloon that inflates on one side of the catheter. Areas where the balloon does not extend around the circumference of the shaft may be used for other purposes, such as placement of an energy source or for solder application. In other embodiments an energy source is placed within a balloon that is transparent to the energy radiating from the energy source.

In the embodiments shown in FIGS. 9 and 10, one energy source 140 is employed. In other embodiments, multiple energy sources (e.g., arrays of LED's, optical fibers, etc.) may be deployed. For example, multiple energy sources may be deployed such that energy is emitted around the circumference of the catheter.

In other embodiments, the catheter shaft is configured to accommodate an energy source, and at least a portion of the catheter shaft is formed of a material that is transparent to the energy radiating from the energy source. In certain embodiments, the catheter may further include a balloon, at least a portion of which is transparent to the energy radiating from the energy source. For example, an elongated member (e.g., a rod or shaft) may be provided with an energy source that directs energy outwardly from the side of the elongated member. When inserted into the catheter, energy can be directed radially outward from the side of the catheter. In certain cases, the transparent material extends completely around the circumference of the catheter shaft (and the balloon in some embodiments). In certain cases, the energy source is rotatable (e g , manually or mechanically), allowing energy to be directed from the catheter in a full circle (i.e., 360° irradiation). This may be also achieved, for example, using an elongated member that directs energy radially from around its entire circumference (e.g., by means of multiple LED's, multiple optical fibers, etc.).

In other embodiments, a catheter may be provided which has a lumen that opens from a distal end of the catheter, allowing an energy source to be extended out of the distal end of the catheter. For example, as shown in FIG. 11, a catheter 150 is shown, which includes a catheter shaft 150s and a balloon 150b. A lumen extends through the catheter and opens at a distal end of the catheter. An elongated member 155 with an energy source 140 at its distal end 155d is inserted through the catheter 150 and extends from the lumen at the distal end 150d of the catheter 150. This embodiment allows the balloon 150 to be withdrawn independently of the energy source 140. In the embodiment shown, the balloon 150b is in the shape of a ring (e.g., having a doughnut shape), although of balloon shapes such as cylindrical, spherical and spheroidal (e.g., prolate spheroidal, oblate spheroidal) balloon shapes may be employed.

As previously seen, in various embodiments, the catheter can be used to deliver a bonding material. For example, catheters can be used to deliver a solid bonding materials such as the sleeves discussed in conjunction with FIGS. 6A-6K, 7A-7F, 8A and 8B. In other embodiments, a balloon may be coated with a bonding material in a fluid form (e.g., a liquid, gel or paste form), and the bonding material may be applied onto the interior urethral wall by expanding the balloon. In other embodiments, a film of bonding material may be placed on a balloon. When the balloon inflates, the film adheres to the interior urethral wall.

In still other embodiments, a catheter may be provided which expels (e.g., sprays, squirts, extrudes, etc.) a bonding material in a fluid form (e.g., a liquid, gel or paste form) such that it contacts an internal surface of a urethral wall. For example, turning to FIG. 12, a dual lumen catheter 150 is shown which contains two balloons. An inner balloon 150bi is provided, which can be inflated by introducing an inflation fluid through an inflation port 150pi. An outer balloon 150bo is provided, which contains pores 150bop. A bonding material in fluid form may be introduced through the fluid port 150pf, which flows into the outer balloon 150b, and out of the pores 150bop. An energy source may be positioned, for example, immediately behind the location where the fluid bonding material is applied such that the bonding material is cured, among various other options.

Thus, as seen from the preceding discussion, the present disclosure describes methods of attaching the first and second urethral portions 110a, 110b to one another using tissue bonding, which may save significant amounts of time and provide an improved seal, relative to methods in which the first and second urethral portions 110a, 110b are attached using sutures. This is advantageous, for example, in that the time period in which a catheter is needed post-operation may be reduced, or the need for a catheter may be eliminated entirely.

It is further noted that tissue bonding can be used in conjunction with a traditional methods in which the first and second urethral portions are joined using mechanical fasteners such as sutures, staples and so forth. In this regard, a mechanically attached urethra may be sealed by placing bonding material at the interface between the first and second urethral portions, internally and/or externally to the urethra. As described above, the bonding material may be in a variety of forms. For example, a ring or hollow tube of bonding material, either in fluid for or in solid form, and either with or without an associated support structure, may be placed in contact with the interface between the first and second urethral portions. An energy source may then be used to supply energy (e.g., heat and/or light) to the bonding material, internally and/or externally to the urethra, activating the bonding material such that the first and second urethral portions are further bonded and/or sealed to one another.

In the above-described procedures, urethral anastomoses are performed.

However, as previously noted, the present disclosure is not limited to urethral anastomoses, and a procedure for esophageal anastomosis will now be described. Esophageal anastomosis may be beneficial, for example, in that it may provide a way for patients with dysphagia due to esophageal cancer to maintain nutrition via oral intake during treatment or palliation periods, among other benefits.

In such procedures, a bonding material may be delivered into the esophagus either before or after the resection. As previously described, the bonding material may comprise, for example, a solder material such as collagen, chitosan, fibrinogen and/or albumin, among other possibilities, which may be mixed, for example, with a photoactivatable chromophore, such as rose Bengal, indocyanine green, methylene blue, riboflavin, SPIONS and/or gold nanorods, among other possibilities. The bonding material may be, for example, in various forms such as those previously described, including the form of a liquid film, a solid scaffold (e.g., a sheet or sleeve), or as a coating on a support structure such as an expandable stent (which may or may not remain after the procedure and which may or may not be bio-absorbable). After the resection, the two esophageal portions may be brought together (or nearly together with a small gap), and the bonding material applied to the esophagus where the portions meet, around an inner circumference of the esophagus. For example, when the bonding material is in the form of a sleeve, the sleeve may be expanded into position within the esophagus. An energy source, for example, from an optical fiber, laser, LED and/or RF energy source, among other possibilities, may be introduced into the esophagus and the sleeve irradiated from an interior of the esophagus, causing the sleeve to crosslink with the tissue, forming a water-tight and immediate bond. In certain embodiments, the solder may bioaborb over a period of 5 to 30 days, among other possible timeframes.

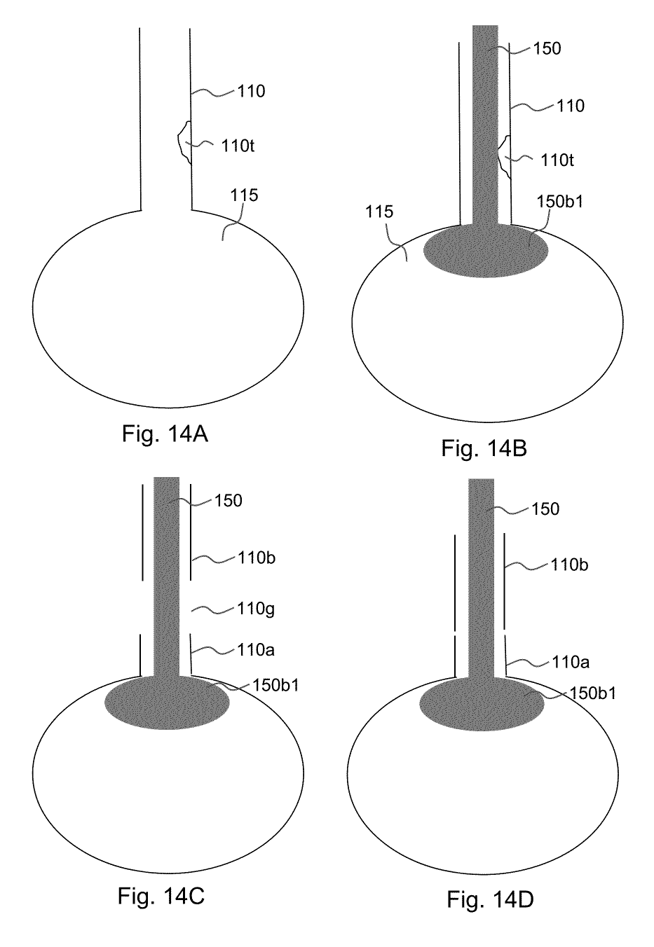

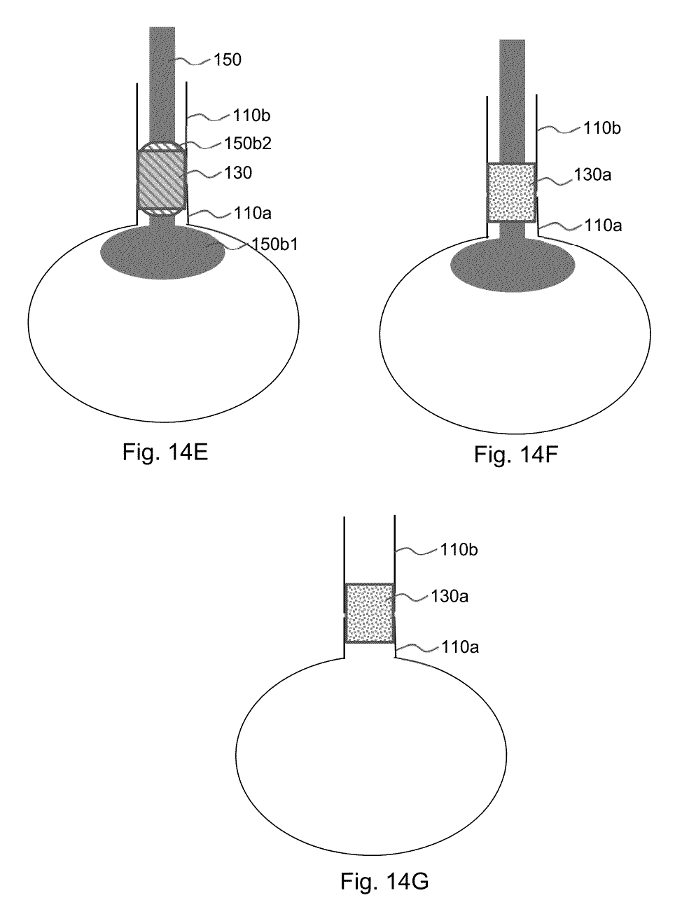

Turning now to FIG. 14A, there is shown therein a stomach 115 and esophagus 110 of a subject having an esophageal tumor 110t. In the embodiment show, a catheter 150 with a first balloon 150b1 is inflated in the stomach 115 to properly position the catheter 150 in the esophagus 110 as shown in FIG. 14B. The catheter 115 may further be provided with a blade (not shown), which can be extended from the catheter to perform the resection and subsequently retracted back into the catheter. In the embodiment shown, the esophageal tumor 110t is resected, thereby forming a first esophageal portion 110a and a second esophageal portion 110b separated by a gap 110g as shown in FIG. 14C. The balloon 150b1 may be pulled back such that the gap 110g is reduced or eliminated as shown in FIG. 14D. A sleeve 130 of bonding material is then provided such that it covers the inside circumference of the first and second esophageal portions 110a, 110b at the point where they meet. The sleeve 130 may, for example, be in the form of a sheet or tube, which may be self-expandable or expandable by placing the sleeve 130 on a surface of an expandable second balloon 150b2 as shown in FIG. 14E. After placement, the sleeve 130 is irradiated by an energy source, for example, an energy source integrated or introduced into the catheter 150 or a separate energy source that is introduced after the catheter 150 is removed, with the result being that the bonding material in the sleeve 130 is activated. For example, an energy source integrated within or introduced into the catheter 150 (not shown) may be energized to form an activated bonding material 130a as shown in FIG. 14F (after deflation of the second balloon 150b2). Finally, the catheter 150 is removed to complete the procedure, as shown in FIG. 14G.

In certain embodiments, the bonding material employed in the present disclosure may comprise various additional agents other than those discussed above, including therapeutic agents and imaging agents, among other possible agents. Such agents may be, for example, incorporated throughout the bonding material or may be applied in a coating over the bonding material, among other strategies.

“Therapeutic agents,” drugs,” “bioactive agents” “pharmaceuticals,” “pharmaceutically active agents” and other related terms may be used interchangeably herein. Therapeutic agents may be used singly or in combination.

In certain embodiments, the bonding material of the present disclosure may comprise one or more therapeutic agents, for example, selected from the following, among many others: (a) anti-inflammatory agents including corticosteroids such as hydrocortisone and prednisolone, and non-steroidal anti-inflammatory drugs (NSAIDS) such as aspirin, ibuprofen, and naproxen; (b) narcotic and non-narcotic analgesics and local anesthetic agents (e.g., for purposes of minimizing pain); (c) growth factors (e.g., for purposes of stimulate the healing process); (d) antimicrobial agents including chlorhexidine, triclosan, nitrofurazone, benzalkonium chlorides, silver salts, silver particles, metallic silver and antibiotic agents such as penicillins (e.g., penicillin G, methicillin, oxacillin, ampicillin, amoxicillin, ticarcillin, etc.), cephalosporins (e.g., cephalothin, cefazolin, cefoxitin, cefotaxime, cefaclor, cefoperazone, cefixime, ceftriaxone, cefuroxime, etc.), carbapenems (e.g., imipenem, metropenem, etc.), monobactems (e.g., aztreonem, etc.), carbacephems (e.g., loracarbef, etc.), glycopeptides (e.g., vancomycin, teichoplanin, etc.), bacitracin, polymyxins, colistins, fluoroquinolones (e.g., norfloxacin, lomefloxacin, fleroxacin, ciprofloxacin, enoxacin, trovafloxacin, gatifloxacin, etc.), sulfonamides (e.g., sulfamethoxazole, sulfanilamide, etc.), diaminopyrimidines (e.g., trimethoprim, etc.), rifampin, aminoglycosides (e.g., streptomycin, neomycin, netilmicin, tobramycin, gentamicin, amikacin, etc.), tetracyclines (e.g., tetracycline, doxycycline, demeclocycline, minocycline, etc.), spectinomycin, macrolides (e.g., erythromycin, azithromycin, clarithromycin, dirithromycin, troleandomycin, etc.), and oxazolidinones (e.g., linezolid, etc.), (e) pharmaceutically acceptable salts, esters and other derivatives of the foregoing, and (f) combinations of two or more of the foregoing.

Additional agents for use in conjunction with the bonding material of the present disclosure include imaging agents such as (a) contrast agents for use in connection with x-ray fluoroscopy, including metals, metal salts and oxides (particularly bismuth salts and oxides), and iodinated compounds, among others, (b) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), and (c) contrast agents for use in conjunction with magnetic resonance imaging (MRD, including contrast agents that contain elements with relatively large magnetic moment such as Gd(III), Mn(II), Fe(III) and compounds (including chelates) containing the same, such as gadolinium ion chelated with diethylenetriaminepentaacetic acid.

In various embodiments, the bonding material may contain from less than 1 wt % to 50 wt % or more of one or more of the preceding additional agents.

In other aspects of the disclosure, medical kits useful in performing an anastomosis are provided. The medical kits may include all or a subset of all the components useful for performing an anastomosis. For example, the medical kits may comprise any combination of any two, three, four, or more of the following items: (a) a support structure as described herein, which may or may not comprise a bonding material, (b) a bonding material in accordance with the present disclosure, for example, in fluid form (e.g., liquid, gel, paste, etc.) or in solid form (e.g., in the form of a tape/ribbon or sheet, in the form of a tube, etc.), (c) a device (e.g., a catheter or other conduit device) configured to apply bonding material in fluid or solid form to first and second body conduit portions, (d) a surgical instrument (e.g., one configured to place first and second body conduit portions over a support structure), (e) an energy source (e.g., in a stand-along unit or associated with a catheter or a surgical instrument), (f) suitable packaging material, and (g) printed material with one or more of the following: (i) storage information and (ii) instructions regarding how to implant the surgical bonding material in a subject.

Although various embodiments are specifically illustrated and described herein, it will be appreciated that modifications and variations of the present disclosure are covered by the above teachings and are within the purview of the appended claims without departing from the spirit and intended scope of the invention.

What is claimed is:

1. An anastomosis bonding component comprising a bonding material, wherein the bonding component is adapted to contact, bond and seal a first body conduit portion to a second body conduit portion upon exposure to energy from an energy source.

2. The anastomosis bonding component of claim 1, wherein the anastomosis bonding component is configured such that the anastomosis bonding component is at least partially disposed within the first and second body conduit portions, such that the first and second body conduit portions are at least partially disposed within the bonding component, or such that the anastomosis bonding component is at least partially disposed within the first body conduit portion and the second body conduit portion is at least partially disposed within the anastomosis bonding component.

3. The anastomosis bonding component of claim 1, wherein the anastomosis bonding component is in the form of a generally tubular member.

4. The anastomosis bonding component of claim 1, wherein the anastomosis bonding component is in the form of a support structure that is configured to support the first and second body conduit portions during an anastomosis procedure.

5. The anastomosis bonding component of claim 4, wherein the support structure is in the form of a hollow tube, solid cylinder, or inflatable cylinder.

6. The anastomosis bonding component of claim 4, wherein the support structure is formed of bonding material or wherein the support structure comprises an underlying structure and a layer of bonding material disposed over all or a portion of the underlying structure.

7. The anastomosis bonding component of claim 1, wherein the anastomosis bonding component is in the form of a generally tubular structure having a diameter that is configured to be reduced after placement around the first and second body conduit portions.

8. The anastomosis bonding component of claim 1, wherein the anastomosis bonding component is in the form of a generally tubular structure having a lumen that extends through the tubular structure, a first end that is adapted to receive the first body conduit portion and a second end that is adapted to receive the second body conduit portion, and wherein a width of the lumen decreases as one moves axially toward the center of the structure from each end.

9. The anastomosis bonding component of claim 1, wherein the bonding material comprises a tissue solder, wherein the bonding material comprises a photosensitizing dye, or wherein the bonding material comprises a tissue solder and a photosensitizing dye.

10. A method of performing an anastomosis procedure comprising: (a) placing a bonding component comprising a bonding material in contact with a first body conduit portion and a second body conduit portion and (b) applying energy from an energy source onto said bonding component, said first body conduit portion and said second body conduit portion, such that the bonding material is activated and the first and second body conduit portions become directly or indirectly bonded to one another and such that a fluid seal is established between the first and second body portions.

11. The method of claim 10, wherein the bonding component is at least partially disposed within the first and second body conduit portions, wherein the first and second body conduit portions are at least partially disposed within the bonding component, or wherein the bonding component is at least partially disposed within the first body conduit portion and the second body conduit portion is at least partially disposed within the bonding component.

12. The method of claim 10, wherein the bonding component is in the form of a fluid bonding component, in the form of a film bonding component, in the form of a support structure formed of bonding material, or in the form of a support structure that comprises an underlying structure and a layer of bonding material disposed over all or a portion of the underlying structure.

13. The method of claim 10, wherein energy is applied from outside the first and second body conduit portions.

14. The method of claim 10, wherein energy is applied from within the first and second body conduit portions.

15. The method of claim 10, wherein the bonding component is delivered by a catheter.

16. The method of claim 15, wherein the catheter further comprises an energy source.

17. The method of claim 10, wherein an amount of energy applied by the energy source is monitored with a temperature sensor and is adjusted based on feedback from the temperature sensor.

18. A kit comprising a combination of any two or more of the following items: (a) an anastomosis bonding component comprising a bonding material, (b) an energy source, (c) a first surgical instrument configured to hold and position a portion of a body conduit adjacent to another portion of said body conduit, and (d) a second surgical instrument that is configured to deliver (i) the bonding component, (ii) energy or (iii) both, to a subject.

19. The kit of claim 18, wherein the second surgical instrument is a catheter selected from a fluid delivery catheter, an expandable catheter, and an expandable fluid delivery catheter.

20. The kit of claim 19, wherein the catheter comprises said energy source.
